(19) United States
(12) Patent Application Publication
(10) Pub. No.: US 2025/0304621 A1
(43) Pub. Date: Oct. 2, 2025

(54) POLYPEPTIDE FOR SPECIFICALLY BLOCKING BINDING OF ACVR1C TO GREM1 AND USE THEREOF

(71) Applicant: The Seventh Affiliated Hospital, Sun Yat-Sen University, Shenzhen (CN)

(72) Inventors: Ningning LI, Shenzhen (CN); Huaixiang ZHOU, Shenzhen (CN)

(73) Assignee: The Seventh Affiliated Hospital, Sun Yat-Sen University, Shenzhen (CN)

(21) Appl. No.: 18/773,646

(22) Filed: Jul. 16, 2024

(30) Foreign Application Priority Data
Mar. 26, 2024 (CN) 202410357923.6

(51) Int. Cl. C07K 7/08; A61K 38/00; A61P 35/00

(52) U.S. Cl. CPC C07K 7/08; A61P 35/00; A61K 38/00

(57) ABSTRACT

A polypeptide specifically blocking the binding of ACVR1C to GREM1 is provided. The small-molecule polypeptide interferes with or blocks the binding of GREM1 to ACVR1C in vitro and in vivo. The liver metastasis of colon cancer in mice in a treatment group is significantly lowered, which indicates that the small-molecular polypeptide achieves the blocking of the liver metastasis of colon cancer.

### CROSS REFERENCE TO THE RELATED APPLICATIONS

This application is based upon and claims priority to Chinese Patent Application No. 202410357923.6, filed on Mar. 26, 2024, the entire contents of which are incorporated herein by reference.

### SEQUENCE LISTING

The present application contains a Sequence Listing which has been submitted in XML format via EFS-Web and is hereby incorporated by reference in its entirety. Said XML copy is named GBRZBC204_Sequence_Listing_20240624.xml, created Jun. 24, 2024, and is 7,579 bytes in size.

### TECHNICAL FIELD

The present invention relates to the technical field of biomedicines, and particularly to a polypeptide for specifically blocking binding of ACVR1C to GREM1 and use thereof.

### BACKGROUND

Colorectal cancer (CRC) is the third most common cancer in the world, ranking third in male cancer incidence and second in female cancer incidence. Colorectal liver metastasis is very common and is also the leading cause of death in patients with colorectal cancer. 20% to 25% of patients with colorectal cancer are accompanied by liver metastasis during initial diagnosis; and after radical resection of the primary lesion, the incidence of metachronous liver metastasis is about 30%, that is, about 50% of patients will eventually develop liver metastasis in the course of colorectal cancer. Meanwhile, due to the characteristics of chronic cancer diseases, cancer patients face high medical expenses and medical resource requirements for many years, and the cancer brings pain to a family and affects the economy of the family and the region. Therefore, the development of specific and effective targeted drugs can effectively prolong the survival of patients with colon cancer metastasis, reduce the economic burden on families, and promote regional development.

For patients with advanced cancer metastasis, chemotherapy drugs still dominate the treatment of tumors, and various side effects caused by radiotherapy and chemotherapy in the traditional treatment schemes are unbearable. The development of specific and effective anti-cancer targeted drugs is of great significance to improving the quality of life and prolonging the survival of cancer patients. However, most of these targeted drugs are “exclusive drugs” that are still under patent protection by foreign pharmaceutical companies and lack competing products. The targeted drugs are often expensive, with monthly drug costs easily reaching tens of thousands or even 100,000 yuan, forcing many patients and their families who cannot afford the drug cost to give up treatment.

The secreted protein Gremlin1 (GREM1), generally considered as a bone morphogenetic protein (BMP) antagonist, plays a crucial role in the pathogenesis and progression of various diseases. Recently, GREM1 has attracted attention due to its cytokine-like properties with affinity binding to a putative receptor. In addition, GREM1 marks a fibroblast subpopulation in normal intestinal and colorectal cancer (CRC). Such GREM1 stromal cells are cancer-associated fibroblasts (CAFs) that promote CRC metastasis.

Activin receptor type-1C (ACVR1C), also known as activin receptor-like kinase 7 (ALK7), is a single-transmembrane type I protein, the extracellular domain of which functions as a receptor and the intracellular domain of which functions as a kinase. Upon ligand binding, a receptor complex is formed consisting of 2 molecules of type II and 2 molecules of type I transmembrane serine/threonine kinases, type II receptors phosphorylate and activate type I receptors, which are automatically phosphorylated and then bind to and activate SMAD transcription regulators SMAD2 and SMAD3. Data from researchers at the Johns Hopkins University School of Medicine support a role of the ACVR1C/SMAD2 pathway in promoting tumor invasion and growth.

ACVR1C is a type I receptor of TGF-β family. Currently, more than 10 TGF-β blocking drugs are being used in combination with PD-1 monoclonal antibodies for clinical trials at home and abroad. So far, no drug has been approved for clinical use, which is probably because most drugs are SMAD pathways downstream of the signaling pathway of this family. The signal transduction downstream of this pathway has critical physiological function of regulating the entire life cycle of living organisms. Therefore, the downstream blockers of this family always suffer from two fatal shortcomings, namely poor specificity and strong side effects.

Therefore, there is an urgent need for further development of specific and effective anti-cancer targeted drugs.

### SUMMARY

In order to achieve the above objective, the present invention provides the following technical solutions.

The present invention provides use of ACVR1C as a receptor of GREM1 in preparing a drug for regulating colorectal cancer.

The present invention further provides a polypeptide specifically blocking the binding of ACVR1C to GREM1, wherein an amino acid sequence of the polypeptide is set forth in SEQ ID NO: 7.

The present invention further provides use of the polypeptide in preparing a drug for treating colorectal cancer.

The present invention has the main advantage that preliminary studies have found that activation of the ACVR1C receptor by GREM1 ligand will significantly promote the colorectal cancer metastasis. The inventors further found the binding site of ACVR1C to GREM1 ligand using a large number of experiments, which is mainly concentrated in an amino acid sequence (TECCFTDFCNNITLHLPTA, SEQ ID NO: 1). Therefore, this amino acid sequence is synthesized, and GREM1 ligand is competitively neutralized, so that the binding of ACVR1C receptor to GREM1 ligand is specifically blocked, the activation of a downstream path is inhibited, and finally the treatment effect of inhibiting colorectal cancer cells metastasis is achieved.

According to the present invention, colorectal cancer cells (HCT116) are injected into NOG mice through spleens, a colorectal liver metastasis model is constructed, and tail vein injection is used for treatment. The results show that the polypeptide significantly inhibits liver metastasis of colorectal cancer cells. It indicates that the polypeptide provided by the present invention is expected to be a simple, safe and effective new drug for preventing and treating colorectal cancer.

### BRIEF DESCRIPTION OF DRAWINGS

FIG. 1 shows the result of the pull-down experiment in Example 1, in which GREM1 binds to surface receptors on the plasma membrane of CRC cells.

FIG. 2 shows the results of mass spectrometry in Example 1, in which four trypsin fragments are identified.

FIG. 3 shows the immunoblot result of the co-immunoprecipitation experiment of Ha-tagged GREM1 in Example 2.

FIG. 4 shows the immunoblot result of the co-immunoprecipitation experiment of Flag-tagged ACVR1C in Example 2.

FIG. 5 shows the co-localization result of ACVR1C and GREM1 in SW480 cells shown by confocal microscopy in Example 2.

FIG. 6 shows the result of the pull-down experiment in Example 2, in which there is a direct physical association between ACVR1C and GREM1.

FIG. 7 shows the co-TP experimental results of the truncated GREM1 and ACVR1C extracellular domain (ACVR1C-ECD) in Example 3.

FIG. 8 shows the potential docking pattern between GREM1 and ACVR1C simulated in Example 3.

FIG. 9 shows the binding between GREM1 and ACVR1C after the corresponding Q72A, E85A or T101A mutations in ACVR1C in Example 3.

FIG. 10 shows the binding between GREM1 and ACVR1C after the Q101A/T102A/T112A/N115A mutations in GREM1 in Example 3.

FIG. 11 is a simulation diagram (a) of the polypeptide blocking the binding of GREM1 to ACVR1C and shows the result of the pull-down experiment in Example 4.

FIG. 12 shows the effect of the polypeptide in treating colorectal liver metastasis in Example 5, wherein a is a live imaging image of a small animal, b is a bioluminescence image of tumor cells in the liver, and c is a statistical graph of liver metastasis luminescence.

### DETAILED DESCRIPTION OF EMBODIMENTS

The technical solutions provided by the present invention will be described in detail below with reference to examples, which, however, should not be construed as limiting the scope of the present invention.

### Example 1

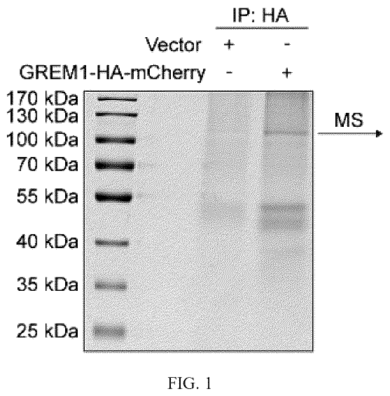

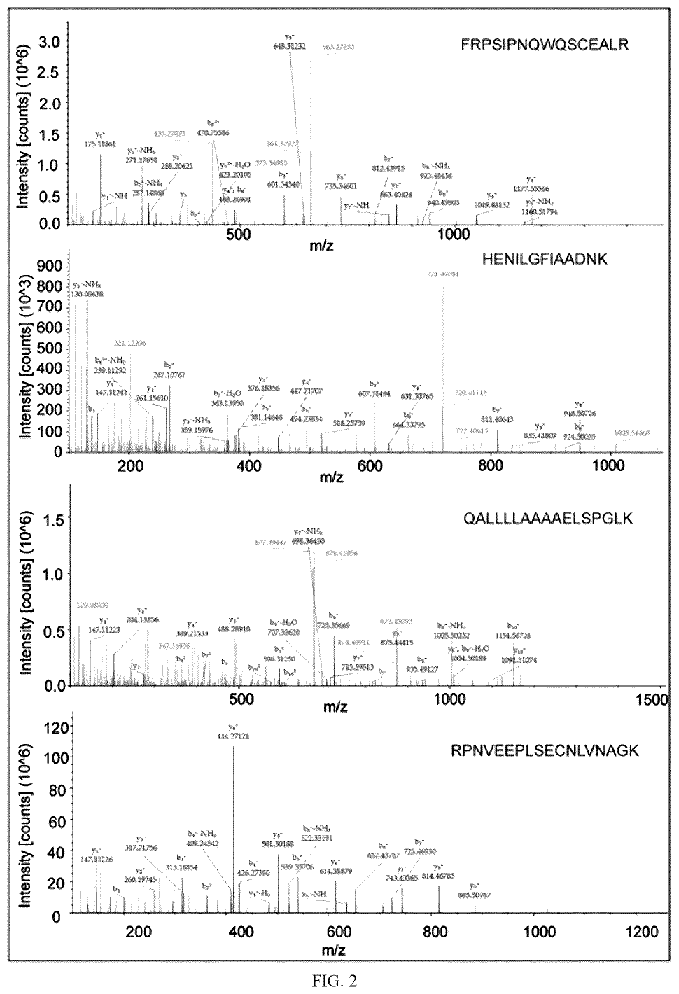

To explore whether GREM1 interacts with surface receptors on the plasma membrane of CRC cells, a pull-down experiment was performed on total protein extracted from human colorectal cancer cells HCT116 transfected with Ha-tagged GREM1 using magnetic beads bound to anti-HA antibodies. The results are shown in FIG. 1, and it can be seen that GREM1 interacts with surface receptors on the plasma membrane of CRC cells. The protein at the fragment indicated by the arrow in FIG. 1 was then subjected to mass spectrometry. The mass spectrometry results are shown in FIG. 2, and four trypsin fragments were identified:

(SEQ ID NO: 1)
FRPSIPNQWQSCEALR
(SEQ ID NO: 2)
HENILGFIAADNK
(SEQ ID NO: 3)
QALLLLAAAAELSPGLK
(SEQ ID NO: 4)
RPNVEEPLSECNLVNAGK.

These four peptides belong to the ACVR1C protein, indicating that ACVR1C is one of the proteins that interact with GREM1.

### Example 2

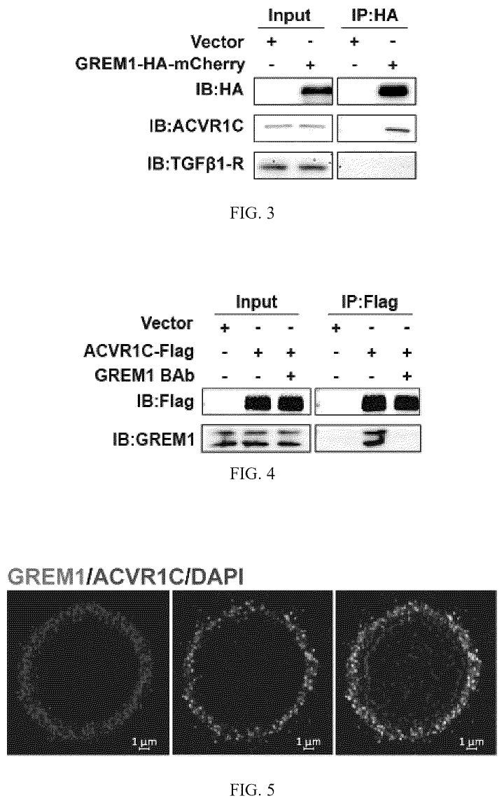

To determine whether ACVR1C is a novel receptor for GREM1, co-immunoprecipitation (co-IP) assays were performed using Ha-tagged GREM1 in HCT116 cells. Subsequent immunoblot results showed an interaction between GREM1 and ACVR1C (FIG. 3). Similarly, co-IP analysis using Flag-tagged ACVR1C indicated an interaction between ACVR1C and GREM1 (FIG. 4). In addition, confocal microscopy showed co-localization of ACVR1C and GREM1 in SW480 cells, which confirmed an interaction between the two proteins (FIG. 5).

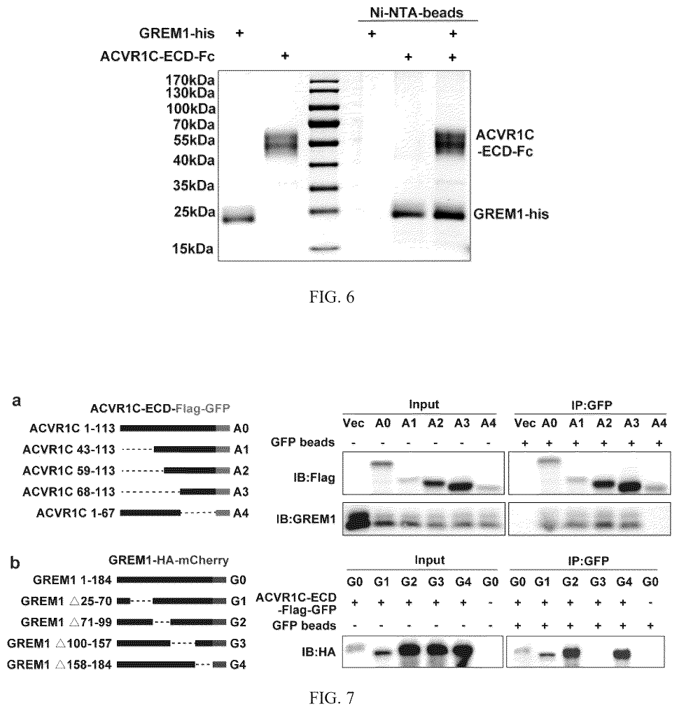

Next, whether there is a direct interaction between GREM1 and ACVR1C is investigated. Therefore, an Fc-tagged ACVR1C extracellular domain (ACVR1C-ECD, AA 1-113, SEQ ID NO: 5) and a his-tagged full-length GREM1 (SEQ ID NO: 6) are purified, and a pull-down experiment is performed, so that the direct interaction between the ACVR1C and the GREM1 is proved (FIG. 6).

ACVR1C (uniport: Q8NER5), ACVR1C extracellular
domain (ACVRIC-ECD) amino acid sequence
(SEQ ID NO: 5):
MTRALCSALRQALLLLAAAAELSPGLKCVCLLCDSSNFTCQTEGA
CWASVMLTNGKEQVIKSCVSLPELNAQVFCHSSNNVTKTECCFTD
FCNNITLHLPTASPNAPKLGPME
GREM1 (uniport: 060565) amino acid
sequence (SEQ ID NO: 6):
MSRTAYTVGALLLLLGTLLPAAEGKKKGSQGAIPPPDKAQHNDSE
QTQSPQQPGSRNRGRGQGRGTAMPGEEVLESSQEALHVTERKYLK
RDWCKTQPLKQTIHEEGCNSRTIINRFCYGQCNSFYIPRHIRKEE
GSFQSCSFCKPKKFTTMMVTLNCPELQPPTKKKRVTRVKQCRCIS
IDLD

### Example 3

To further describe the binding sites between GREM1 and ACVR1C, a truncated GREM1 and ACVR1C extracellular domain (ACVR1C-ECD) is constructed. Subsequently, co-IP experiment showed that the absence of amino acids 100-157 of GREM1 (AA 100-157, FIG. 7b, G3) or amino acids 68-113 of ACVR1C-ECD (AA68-113, FIG. 7a, A4) effectively abolished the interaction between GREM1 and ACVR1C in HCT116 cells.

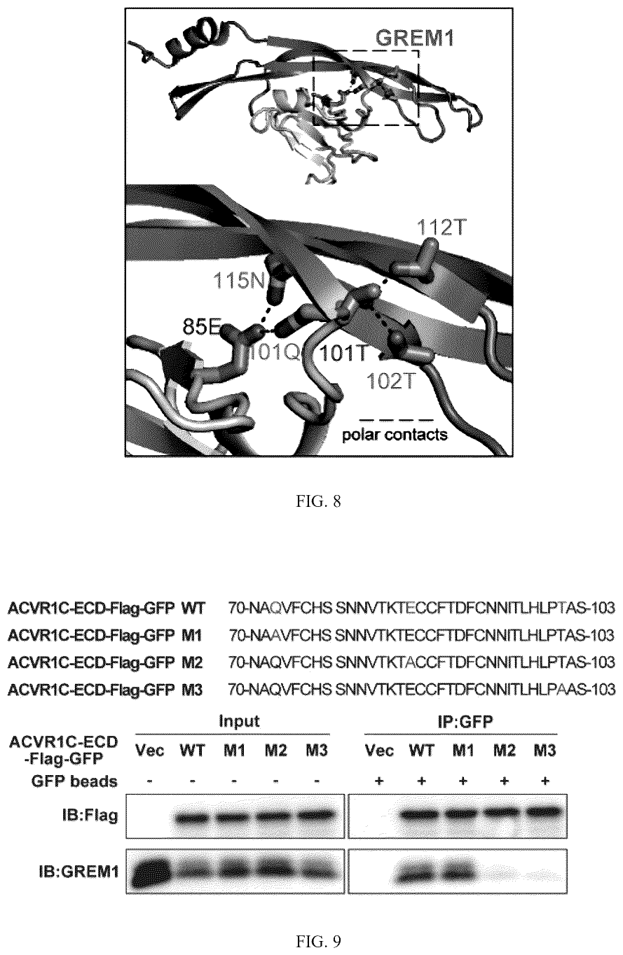

Based on this result, the key interaction sites between GREM1 and ACVR1C are further identified. The potential docking pattern between GREM1 (PDB: 5AEJ) 34 and ACVR1C (AlphaFold prediction, alphafold.ebi.ac.uk) were simulated using the HDOCK platform (hdock.phys.hust.edu.cn) according to their protein structures. The amino acid residues Q101/T102/T112/N115 in GREM1 and amino acid residues Q72/E85/T101 in ACVR1C were predicted to be essential binding regions (FIG. 8).

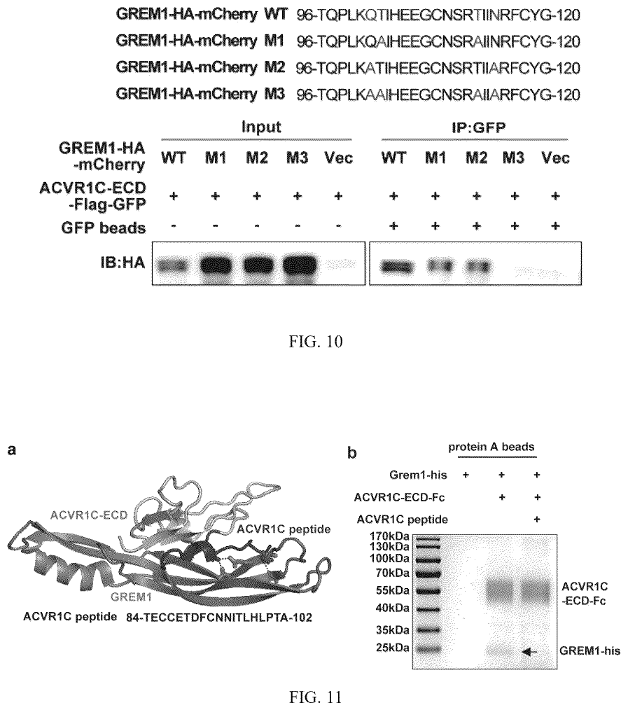

For validation, mutagenesis was performed according to the potential sites described above to test protein binding simulation. It should be noted that the quartet mutations of Q101A/T102A/T112A/N115A in GREM1 or the corresponding E85A or T101A mutations in ACVR1C severely disrupted the association between GREM1 and ACVR1C (FIGS. 9 and 10).

### Example 4

According to the ligand relationship between ACVR1C and GREM1 determined and the key binding sites mined in Examples 1 to 3, a peptide inhibitor based on ACVR1C amino acid sequence 84-102 (AA 84-102) was designed, referred to as ACVR1C peptide.

The Fc-tagged ACVR1C extracellular domain (ACVR1C-ECD, AA 1-113) and his-tagged full-length GREM1 were purified, and ACVR1C peptide (with an amino acid sequence of TECCFTDFCNNITLHLPTA, SEQ ID NO: 7) which blocked the binding of ACVR1C to GREM1 ligand thereof was synthesized (FIG. 11a). The Pull-down experiment was performed. The results showed that the ACVR1C peptide competitively inhibited the binding of GREM1 to ACVR1C (FIG. 11b).

### Example 5

Grouping: a total of 18 6-week-old NOG mice were taken and randomized into 3 groups 5 days after adaptive feeding: control group (PLV), GREM1 overexpression group (PLV-GREM1) and ACVR1C polypeptide treatment group (PLV-GREM1+ACVR1C peptide), 6/group.

Modeling: HCT116-luc cell suspensions were prepared with cell densities adjusted to 1×10⁷ cells/mL. The mice to be treated were taken out and anesthetized by intraperitoneal injection of 2% sodium pentobarbital at a dose of 45 μL/20 g. After about 5 minutes, the mice began to enter a quiet state. White gauze was spread on a mouse board, the mice were placed in left lying position, the abdominal hair of the mice was shaved, and the abdominal side was disinfected from the neck to the groin with alcohol cotton pads. The skin was cut open 1-2 cm below the lower edge of the left costal arch to expose the abdominal wall muscles. The abdominal wall muscles were pulled with pointed forceps to avoid the spleen in the abdominal cavity. The abdominal wall muscles were cut open to expose the internal organs. The pancreas was gently pulled with circular forceps, and the spleen was pulled out. 50 μL of single cell suspension was aspirated from the EP tube by an insulin syringe, the needle core was slightly pushed, after air bubbles were removed, the cells were injected into the spleen, the needle was slowly withdrawn to prevent cell leakage, and pressure was applied to stop bleeding.

Administration: the administration concentration of the polypeptide was 10 mg/kg, and the injection was administered by tail vein once every 2 days. The control group and GREM1 overexpression group were given equal amounts of PBS (both by tail vein injection).

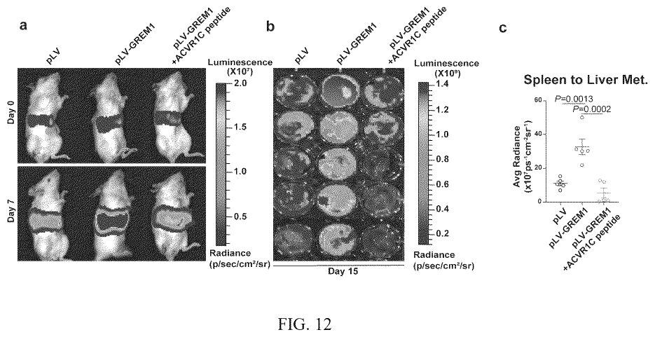

Observation: the growth and metastasis of tumors were observed using a small animal live imaging system. Before the start of the experiment, 150 mg/kg luciferase (diluted with DPBS without Mg²⁺ and Ca²⁺) was injected into each NOG mouse, after about 10 minutes, the NOG mice were anesthetized with isoflurane and observed by live imaging, and bioluminescence images were taken, as shown in FIG. 12. The animal experiment results show that the polypeptide can significantly inhibit the colorectal cancer metastasis.

It can be known from the above example that the present invention discovers a new ligand-receptor relationship between GREM1 and ACVR1C and determines the binding sites between GREM1 and ACVR1C; and then designs a small-molecule polypeptide (ACVR1C peptide) for specific blocking based on the details of the binding of GREM1 to ACVR1C. The small-molecule polypeptide can interfere or block the binding of GREM1 to ACVR1C in an in vitro experiment; and an in vivo treatment experiment of the small-molecule polypeptide shows that the liver metastasis of colon cancer of mice in a treatment group is significantly lower than that in a control group, which indicates that the small-molecular polypeptide achieves the blocking of the liver metastasis of colon cancer by interfering or blocking the binding of GREM1 to ACVR1C, and provides a new drug design and a new therapeutic target for relieving the liver metastasis of colon cancer.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A method for preparing a drug for regulating colorectal cancer comprising the step of using ACVR1C as a receptor of GREM1.

2. A polypeptide specifically blocking binding of ACVR1C to GREM1, wherein an amino acid sequence of the polypeptide is set forth in SEQ ID NO: 7.

3. A method for treating colorectal cancer comprising the step of administering an effective dose of the polypeptide according to claim 2 to a subject suffering from colon cancer.


SEQUENCE LISTING (7 sequences)

SEQ ID NO: 1 - protein, 16 aa, synthetic construct
FRPSIPNQWQSCEALR

SEQ ID NO: 2 - protein, 13 aa, synthetic construct
HENILGFIAADNK

SEQ ID NO: 3 - protein, 17 aa, synthetic construct
QALLLLAAAAELSPGLK

SEQ ID NO: 4 - protein, 18 aa, synthetic construct
RPNVEEPLSECNLVNAGK

SEQ ID NO: 5 - protein, 113 aa, synthetic construct
MTRALCSALRQALLLLAAAAELSPGLKCVCLLCDSSNFTCQTEGACWASVMLTNGKEQVIKSCVSLPELNAQVFCHSSNNVTKTECCFTDFCNNITLHLPTASPNAPKLGPME

SEQ ID NO: 6 - protein, 184 aa, synthetic construct
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 7 - protein, 19 aa, synthetic construct
TECCFTDFCNNITLHLPTA